(19) United States
(12) Patent Application Publication
(10) Pub. No.: US 2006/0154248 A1
(43) Pub. Date: Jul. 13, 2006

(54) Manufacturing method and readout system for biopolymer arrays

(76) Inventors: Stephen P McGrew, Spokane, WA (US); Donald R Lightfoot, Spokane, WA (US)

Correspondence Address: PERKINS COIE LLP, P.O. BOX 2168, MENLO PARK, CA, 94026, US

(21) Appl. No.: 10/529,834

(22) PCT Filed: Oct. 2, 2003
(86) PCT No.: PCT/US03/31490

§ 371 Date: Oct. 24, 2005

Related U.S. Application Data
(60) Provisional application No. 60/415,542, filed on Oct. 2, 2002.

(51) Int. Cl. C12Q 1/68; C12M 1/34; B01L 3/02; G01N 27/00

(52) U.S. Cl. 435/6; 435/287.2; 435/288.7; 422/100; 427/2.13; 422/82.01; 436/151

(57) ABSTRACT

A method for manufacturing an array of biomolecules that have complementary binding species is disclosed. The method utilize a master substrate containing a desired master array of binding molecules or ligands, e.g., DNA target molecules. A mixture of complementary, antiligand molecules, e.g., DNA probe molecules, are then hybridized to the ligands of the master array, Forming an array of antiligand molecules reversibly bound to the molecules in the master array. The antiligand array is then transferred and bound to a separate substrate, to form a desired array of antiligand molecules on the separate substrate. The process may be repeated to produce multiple arrays or multiple generations of arrays. Also disclosed is a sensor array device and method. The device includes an array of vibratory tympanic elements, each having a specific analyte binding probe carried on the vibratory membrane of the element. A binding event is detected by a change in the vibrational resonance of a selected element.

### SUMMARY OF THE INVENTION

A method is described for manufacturing DNA arrays and the like, using a hybridization transfer printing process to replicate an original DNA array onto a new kind of sensor array. The hybridization transfer printing produces a reverse replica of an entire DNA array comprising any number of different DNA segments. The sensor array enables direct electrical readout to determine the presence of complementary DNA bound to the DNA array at each element of the sensor array.

### BACKGROUND OF THE INVENTION

DNA arrays and antibody or antigen arrays have become common tools for DNA sequencing, DNA analysis, protein analysis, detection of specific microbial species, disease diagnosis, and other such purposes. See, for example, U.S. Pat. Nos. 6,376,191, 6,368,799, 6,316,197, 6,309,824, 6,309,823, 6,245,511, 6,232,068, 6,027,890, and 5,861,242, 5,856,101, all of which are incorporated herein by reference. The arrays are commonly either built up by an automated robotic dispenser of DNA, antibodies or antigens, or else are “grown” by a photo-controlled DNA synthesis procedure. See, for example, U.S. Pat. Nos. 6,406,851, 6,391,625, 6,387,626, 6,383,,784, 6,326,489, 6,309,831, 6,284,497, 6,251,685, 6,187,537, 6,171,797,, 6136,962, 6,127,129, 6,110,426, 6,083,726, 6,083,697, 6,030,782, 5,919,626, 5,837,832, 5,807,522, 5,688,642, all of which are incorporated herein by reference. Also disclosed in the prior art are various optical and electronic methods for detecting binding of target or analyte molecules to array probes. See, for example, U.S. Pat. Nos. 6,403,317, 6,391,558, 6,355,431, 6,232,068, 5,874,219, 5,843,651, 5,633,724, 5,605,662, and 5,599,688, all of which are incorporated herein by reference. Various reaction conditions by which target or analyte molecules are bound to array probes have also been disclosed. See, for example, U.S. Pat. Nos. 6,258,534, and 6,107,038, which are incorporated herein by reference.

In the photo-controlled DNA synthesis method, in order to grow an array of various 20-nucleotide DNA strands, projected light patterns control the polymerization of DNA in “layers”. Each “layer” requires four main steps and four different projected patterns to specify the particular array elements where As, Gs, Cs or Ts are to be added. Thus, a 20-nucleotide DNA strand array requires 4×20=80 steps regardless of the number of different 20-mers grown on the array. Each step involves substeps such as rinsing, adding reagents, incubation, photo-exposure, and so on. The method is inherently lengthy and expensive, so that individual DNA arrays (either on silicon, glass or plastic) can cost as much as $1200 each.

To use the prior art DNA array chips, a mixture of unknown DNA strands is poured over the array and allowed to hybridize with the DNA strands on the chip. Any DNA in the mixture that is complementary to DNA on the chip hybridizes with DNA at the corresponding array elements on the chip, forming double-stranded DNA. The presence of double-stranded DNA is determined by applying intercalating dyes, for example, and scanning the chip to determine which nodes have become fluorescent. The fluorescence analyzer is expensive and often misreads the locations of individual array elements.

There have been proposals to make DNA chips as arrays of silicon cantilevers, allowing electrical readout. However, such cantilever arrays would have some potential problems of clogging and would be even more expensive to manufacture than the passive arrays commonly used with fluorescence readout

### SUMMARY OF THE INVENTION

In one aspect, the invention includes a method for manufacturing an array of biomolecules that have complementary binding species. In practicing the method, there is formed on a first substrate, a master-species array composed of a plurality of different molecular species (ligand molecules) attached to selected regions (elements) on the first substrate. A mixture containing biomolecules (antiligand molecules) complementary to each of these species is then brought into contact with the master species array, such that biomolecules in the mixture bind selectively and reversibly to complementary species in the master array. A second substrate is then positioned in a confronting relationship with the master array, such that the complementary biomolecules bound to the master-species array are in proximate confronting relationship to corresponding array regions in the second substrate. The bound biomolecules from the first substrate are then transferred to confronting substrate, forming a complementary, mirror-image array of biomolecules on the second substrate.

The antiligand biomolecules may include a binding moiety effective to react with and bind covalently to the surface of the second substrate, when contacted with the second substrate during the transferring step. In one embodiment, the binding biomolecules include a sulfhydryl binding moiety, and the confronting surface of the second substrate includes a gold film.

Biomolecules often carry a charge, so that if a film of electrolyte is placed between between confronting surfaces of the first and second substrates, the transferring step may be carried out by applying between the first and second substrates, an electric field whose polarity and field strength are effective to cause the biomolecules bound to the first substrate to migrate electrophoretically to the proximate corresponding array regions of the second substrate.

For use in forming an array of different-sequence oligo-or polynucleotides, the ligand species on the first substrate may include different-sequence oligonucleotides, where the antiligand biomolecules include oligo- or polynucleotides containing at least a segment that is complementary to one of the ligand species. The oligo- or polynucleotide biomolecules may include a sulfhydryl binding moiety, and the confronting surface of the second substrate may include a gold film to which the sulfhydryl moiety can bind, as a result of the transfer step. Alternatively, the second substrate may be coated with an oligonucleotide having a given sequence, where each of the antiligand biomolecules bound to the first substrate has a segment whose sequence is common to the other biomolecules and complementary to the oligonucleotide coating the second substrate, or a thiol end on the antiligand may be used to bind the antiligand to the second substrate (the receiver substrate).

The method may include repeating the bringing, positioning, and transferring steps multiple times, thereby to produce multiple arrays. The master species array may be in the form of a flat sheet or plate., or it may be in the form of a cylindrical drum and the substrate of the complementary species arrays may be in the form of a flat sheet or plate, or in the form of a continuous roll of film

The master-species array may be one that itself has itself been formed by the method of the invention, to produce a subsequent generation of complementary replica arrays.

The method may be carried out so that at least one of the processes is done on a roll-to-roll basis with at least one of the substrates being unrolled from a feed roller and re-rolled onto a take-up roller.

In one embodiment, the second substrate has an array of vibratory elements formed on the substrate, where each element has a vibratory membrane with a characteristic resonance frequency, when activated by an oscillating electrical field applied to the membrane. The positioning step includes placing the second substrate into a confronting relationship with the master array, such that biomolecules bound to the master-species array are in proximate confronting relationship to corresponding vibratory elements in the second substrate.

A sensor device formed by one embodiment of the invention, includes a substrate having a plurality of array regions, and attached to each array region, an anchoring nucleic acid covalently attached to each region, where the anchoring nucleic acids have a common base sequence. Hybridized to the anchoring nucleic acids in each region is a nucleic acid probe having a first common-sequence region that is complementary to at least a portion of the anchoring nucleic acid, and a second probe region having a nucleic acid sequence that is different in the different regions. The common-sequence region of the probes may be covalently linked to the complementary anchoring nucleic acid, e.g., by UV thymine dimer formation.

In another aspect, the invention includes a sensor array device having a plurality of individually addressable deformable membrane elements having resonant frequencies that change when mass is added to or removed from the membrane in each sensor element. Also included in this device is a readout system that detects changes in the resonant frequency of at least one membrane element in the array, and a signal-processing system for determining, from detected changes in resonance frequency, the presence of a change in mass of said system.

In a more specific aspect, the invention includes a sensor array device for use in detecting the binding of analyte molecules to one or more array probes. The device includes a substrate having an array of drum-like or tympanic vibratory elements formed thereon, where each element has a vibratory membrane having a characteristic resonance frequency, when activated by an oscillating electrical field applied across the membrane. Attached to the membrane of each element array is a molecular species probe, where binding of analyte molecules to a probe is effective to change the mass, and thereby alter the resonance frequency, of the corresponding membrane. Associated with each element is a pair of electrodes for addressing and stimulating the membrane on that element. For example, one electrode coated directly on the membrane and one spaced a small distance away from the membrane, by which a voltage can be applied between the two electrodes. A voltage source is operatively connected to the electrodes for applying an oscillatory voltage to the pair of electrodes associated with a selected element membrane, thereby to induce vibration in the membrane. A detector in the device is designed to detect changes in the frequency or amplitude of induced vibration in an element membrane, thereby to determine mass change corresponding to the presence or absence of analyte bound to each of one or more membranes in the device.

Where the array includes a plurality of rows and plurality of columns of elements, the pairs of electrodes associated the elements may be formed by a plurality of row electrodes positioned on one side of a plurality of rows of elements, and a plurality of column electrodes positioned on the opposite side of a plurality of columns of elements. The elements may be cylindrical cavities in a semi-rigid layer of material on a solid, semi-rigid substrate, with the cavities capped by a flexible membrane. The electrodes may be gold stripes coated onto the membrane along the rows, and gold stripes coated onto the substrate along the columns. In this embodiment, the voltage source may be operable to excite selected row and selected column electrodes, thus to induce vibration in one or more selected elements.

The voltage source may be operable to apply a variable-frequency alternating voltage to a selected element, and the detector may be operable to detect the frequency at which resonance occurs.

For use in detecting the presence or absence of each of a plurality of different DNA analytes, the probes may be different-sequence nucleic acid probes. For use in detecting the presence or absence of each of a plurality of different polypeptide analytes, the probes may be different-polypeptide-binding probes. For example, the probes may be antibodies selected to bind specifically to different antigens.

In a related aspect, the invention provides an optically-addressed hybridization array sensor device that includes an array of vibratory elements each formed of a substantially cylindrical rigid cavity covered by a flexible membrane. The membrane bears an electrically conductive layer and a probe species layer. The bottom of the cavity bears a photo-controlled conductive element and electrical contacts attached to an electrical signal source, such that illumination of the conductive element controls an electrical voltage between the bottom or sides of the cavity and the electrically conductive layer of the membrane, and such that the membrane is deflected by an electrostatic force controlled by the electrical voltage. The general configuration of the sensor in this case is similar to the optically controlled light modulator in FIG. 3, but the function and purpose are different. Also included is an optical addressing system that directs illumination to selected elements of the array at selected modulation frequencies, and a system to detect the response of the drum-like structures to the selected frequencies, for detecting change in an amount of target species hybridized to the probe species.

In still another aspect, the invention includes an optical read-out system for a hybridization array sensor device of the type having an array of deflectable membrane modulators, where the resonant frequency of each membrane modulator is controlled by hybridization of a target species to a probe species on the membrane of the membrane modulator. The read-out system includes a signal source and driver to drive the entire array of modulators at a series of selected frequencies, an optical phase detection system to measure phase differences between the signal source and the deflection of the modulators at the selected frequencies, and an information processing system to relate the phase differences to changes in the hybridization state of the membrane modulator.

Also disclosed is a system for producing chemical arrays on a roll-to-roll basis. This system has a plurality of exposure modules, each comprising a variable masking system, a light source and imaging optics to image a line of pixels from a variable mask to a line on a surface. A plurality of photochemical modules function to bring reactants of a photo controllable chemistry in contact with a substrate bearing reaction loci, such that at each photochemical module is associated with at least one exposure module such that the variable masking system of the exposure module controls illumination of a line of reaction loci on the substrate and thereby controls the photo-controllable chemistry in a spatial pattern of loci according to the variable masking system, such that the plurality of photochemical modules and their associated exposure modules effect an individually predetermined sequence of photo-controlled reactions at each locus on the substrate.

In still another aspect, the invention includes a roll-to-roll DNA array manufacturing system. The system includes a web handling system to move a substrate in the form of a flexible web through a series of groups of four photochemical reactors, where each of the photochemical reactors includes a source of photo-controlled chemistry and an exposure module. The reactive molecules on the substrate are brought into contact with each of four nucleic acids A, G, C, T, in a predetermined order and the exposure module controls the illumination of the substrate at each of a plurality of loci in a line on the substrate substantially perpendicular to the direction of web motion and on the substrate in a predetermined spatial pattern.

These and other objects and features of the invention will become more fully apparent when the following detailed description of the invention is considered in conjunction with the accompanying drawings.

### BRIEF DESCRIPTION OF THE FIGURES

FIG. 1 is a representation of an embodiment of the present invention, including an antigen array “chip” comprising a flexible substrate, longitudinal gold stripe electrodes, a UV-cast perforated layer, a thin elastic membrane with lateral gold stripe electrodes, and antibodies bound to the gold stripe electrodes in the regions directly over the perforations in the UV-cast layer. Note that in the drawing of FIG. 1 both sets of stripe electrodes are uncovered at edges of the “chip” so that electrical contact can be made with the electrodes.

FIG. 2 is a copy of a drawing of Preston's prior art deflectable membrane light modulator (MLM).

FIG. 3 is a copy of a drawing of Reizman's prior art deflectable membrane light modulator.

FIGS. 4a through 4e show an exploded representation of the “chip” of FIG. 1, with electrodes crossing at the positions of perforations in a perforated layer.

FIG. 4a represents DNA molecules of different types, arranged into an array of spots.

FIG. 4b represents an array of spots to which the complementary probe substances to a set of target substances are bound.

FIG. 4c represents an elastic membrane with electrode stripes in one direction under the array of spots in FIG. 4b to which the complementary substances are bound.

FIG. 4d represents a perforated layer with perforations under the array spots, between the electrodes, the perforations located at the crossings of the electrode stripes.

FIG. 4e shows a substrate bearing electrode stripes in a direction perpendicular to the stripes in FIG. 4b, positioned directly under the perforations of FIG. 4c.

FIGS. 5, 6, 7, 8 and 9 illustrate a version of the process for making complementary substance arrays according to this invention.

FIG. 5 represents the step of binding target substances to spots on a substrate.

FIG. 6 represents the step of introducing a mixture of tetherable-end probe substances complementary to the target substances and letting them hybridize with the target substances.

FIG. 7 represents the substrate with various hybridized substances at an array of spots.

FIG. 8 represents the step of bringing a second, derivatized substrate into proximity to the tetherable ends of the complementary substances and binding the tetherable ends to the second substrate.

FIG. 9 represents the step of separating the two substrates, such that the target substances stay bound to the first substrate and the complementary probe substances stay bound to the second substrate.

FIG. 10 represents a prior-art method of depositing substances such as gold or aluminum onto a web by evaporation in a vacuum, following printing of a masking substance in a pattern to allow the deposition to occur only in selected regions.

FIG. 11 illustrates a roll-to-roll process for patterning electrodes on a web. The process includes a photoresist coating step onto a film web pre-coated with an electrically conductive material; a UV exposure step including a UV light source, concentrator lens, variable mask, and projection optics; a photoresist developing step; a rinsing step; registration sensors; and subsystems to feed the film web from a feed roller and accumulate the patterned web onto a take-up roller.

FIG. 12 represents a system for continuously casting a perforated layer onto a film web substrate with the perforations in register with electrodes pre-patterned on the substrate, for laminating a thin elastic membrane onto the perforated layer, and for completing a UV cure of a UV resin used to form the perforated layer.

FIG. 13 illustrates a vacuum deposition system using a fiber mask to deposit substances in the form of longitudinal stripes along the length of a web. Fibers are fed from a spool through guide holes, and registration subsystems ensure that the longitudinal stripes are deposited in register with pre-existing patterns on the web. Continuous feeding of the fibers prevents excessive buildup of gold or other materials on the mask.

FIG. 14 illustrates a roll-to-roll process for “printing” arrays of hybridizable substances onto a web in register with pre-patterned structures on the web. It includes a master array drum, registration sensor, baths for the various chemistries required for hybridization, incubation, separation and rinsing, a dryer, feed roller and takeup roller.

FIG. 15 represents a web bearing hybridization sensors in longitudinal strips on a substrate ready for diecutting into individual array chips.

FIG. 16 illustrates a readout device for hybridization sensor array chips made according to the present invention.

FIG. 17 describes the operation of the readout electronics for a hybridization sensor array.

FIG. 18 illustrates a photo-addressable tympanic array sensor for hybridization of target substances and complementary substances on spots on the sensor, along with an optical system for reading the hybridization states of the spots.

FIG. 19 illustrates a photochemical reactor module in which a variable masking system exposes a line pattern of pixels and thereby produces a spatial pattern of photochemically induced reactions on a substrate.

FIG. 20 illustrates a series of modules of the sort described in FIG. 17, for the purpose of manufacturing a continuous web of DNA or other substance arrays in a series of photo-controlled ligation reactions.

### DETAILED DESCRIPTION OF THE INVENTION

The present invention has two important aspects: First, providing a simple way (Hybridization Transfer Printing) to eliminate the many steps involved in building a DNA, antibody, antigen or microbe array; and a simple electrically-driven sensor array to detect the presence of complementary hybridization to 30 individual elements in the array. The invention uses essentially the same techniques to produce and analyze DNA arrays, microbe arrays, antibody arrays and antigen arrays. By greatly reducing the number of steps to manufacture arrays for specific hybridization and providing a simple and easily manufacturable sensor array, the present invention solves the problems of cost and complexity in DNA chip manufacture and opens a host of new practical applications for hybridization arrays. The second general aspect is a Tympanic Sensor Array that allows electronic detection of multiple binding events occurring on an array.

I. Hybridization Transfer Printing

The inventive method for replicating arrays of DNA strands takes advantage of the well-known fact that complementary strands of DNA bind much more strongly than non-complementary strands to form double-stranded DNA, and that a DNA strand may be made to bind by one end to a substrate such as gold more strongly than to the strand's DNA complement. e.g., by covalent attachment of the strand to the substrate or by binding to a substrate bound molecule with a greater binding affinity, e.g., higher Tm. in a process analogous to molding and casting, the inventive method produces a sequence of “positive” and “negative” copies of an original arrangement of molecular species, with a “positive” copy being essentially an arrangement identical to the original arrangement, and a “negative” being essentially a mirror-image arrangement of molecular species complementary to the corresponding species in the original arrangement. The process of making such copies may be carried out through a series of generations to produce a large number of essentially identical replicas of the first and second generation “positive” and “negative” arrays.

A. Definitions

For convenience, in the following description the terms “ligand” and “anti-ligand” will be used to include any pair of molecules that have specific reversible binding affinity to each other, including complementary nucleic acid molecules, antibody-antigen pairs, receptor-binding molecule pairs, e.g., receptor-protein or receptor-drug pairs. The term “complementary” will be used to indicate a specific binding relationship between a ligand and anti-ligand. For example, a DNA strand consisting of a polymer of A, G, C, and T nucleotides is complementary to another DNA strand having the same sequence of nucleotides except with a T substituted for each A, a C substituted for each G, a G substituted for each C, and an A substituted for each T. Similarly, an RNA strand consisting of U, C, G and A is complementary to a DNA strand in which an A is in the place of each U, a G in the place of C, a C in the place of each G, and a T is in place of each A in the RNA strand. Similarly, an antibody is complementary to its antigen and vice versa. Complementarity is usually, but not necessarily, a reciprocal relationship. For purposes of illustration, the term “DNA” may here be used to represent one of a binding pair, and “complementary DNA” may here be used to represent the other molecule in a binding pair, it being understood that “DNA” may be representative of other ligand-antiligand pairs than deoxyribonucleic acids.

B. Hybridization Transfer Method

In a first step of the hybridization transfer printing method, a first substrate is prepared with an array of regions of gold or other material, e.g., derivatized polymer or glass, to which DNA can be attached, e.g., by covalent attachment.

In a second step, DNA species having different selected sequences are bound to the regions such that each region contains only one kind of “probe” or “target” DNA., that is, such that each region contains only ligand DNA having one particular nucleotide sequence, and each region has its own particular nucleotide sequence. The resulting original DNA array is referred to herein as a “master array”. The master array can have either probe or target DNA depending on the number of generations of replication there will be between the original array and the final arrays. These first two steps are well-known in the art.

In a third step, a mixture of anti-ligand “target” or “probe” DNA strands is hybridized with the DNA on the master array, such that each region of the master array then contains only one kind of target DNA and one kind of probe DNA complementary to the target DNA. The probe DNA molecules in this third step may have an “interface” end or a “tether”: a molecular segment or a nanometer-scale particle bound strongly (e.g., covalently bound) to the end of the DNA molecule and providing a means for attaching the probe DNA molecule to a second substrate. The chemistry of this third step is well known in the art.

In one embodiment, the second substrate contains an anchoring nucleic acid covalently or otherwise tightly bound to each array region in the second substrate. The anchoring nucleic acids have a common sequence in, and may be identical from region to region. In this embodiment, the tether in the different-sequence probes includes a nucleic acid region (a first common-sequence region) that is complementary to the anchoring nucleic, and a second, region-specific probe portion that is different from sequence to sequence. The anchoring nucleic sequence and complementary probe common sequence are preferably selected, either by GC content, length, or backbone structure, to provide a substantially higher Tm of binding than the Tm of the hybridized portion of the probe bound to the master substrate.

During the transfer operation, with the two substrates placed in confronting relationship, the substrates are heated to a temperature above the Tm of the probe, master-substrate nucleic acid but below the Tm of the probe/anchoring nucleic acid in the second substrate, to selectively release the probes from the master copy, transferring the probes to the second substrate. Once the second substrate is removed, the hybridized portion of the nucleic acid probes on the substrate can be covalently attached, e.g., by UV thymine dimer formation with the anchoring nucleic acid.

Alternatively, the anchoring nucleic acid may include reactive groups, e.g., reactive bifunctional groups that are capable of reacting selectively with hydridized portions of the probe, so that the master and second substrate can be separated without regard to the relative Tms of the two hybridized probe portions. In any event, it is preferable during a transfer operation to heat the master substrate above the Tm of the hybridized nucleic acids on the master substrate, to facilitate release of the probes from the master substrate.

In a fourth step, a second substrate is brought into close proximity to the probe DNA tethers and the tethers are caused to bind strongly to the second substrate. This binding to the second substrate can be thiol-gold binding, for example, carried out using well-known chemistry. Although the chemistry is not new, the in situ binding of tetherable ends of hybridized complementary species to a second substrate is novel.

In a fifth step, the two substrates are separated, e.g., at an elevated temperature above the Tm of the probe binding to the master-array molecules, such that the ligand DNA stays attached to the first substrate and the anti-ligand DNA stays attached to the second substrate. The anti-ligand DNA on the second substrate thus constitutes a “complementary DNA array”, complementary to the master array. The anti-ligand DNA array is effectively a complementary mirror image of the master target DNA array. This set of five steps is referred to herein as “hybridization transfer printing”.

It is advantageous to select a second substrate that is flexible or elastic enough to conform to any deviations from flatness in the first substrate Alternatively, the first or second substrate may be made of an elastomeric material, or the DNA on the first substrate may be bound to raised elastomeric “plateaus” to ensure close contact between the surfaces of the first and second substrates when they are pressed together. It is also possible to actively transport the probe DNA to the second substrate by applying a voltage between the two substrates such that the DNA is electrophoretically driven from the first substrate to the second substrate. Binding of the probe DNA to the second substrate is preferably via a covalent bond such as thiol group to gold or an amine, acid, aldehyde, or alcohol group to a suitable chemical group on the second substrate. The covalent attachment may be by the addition of suitable activating agents or pH conditions or bifunctional reagents, according to well-known coupling methods (see below). A voltage may similarly be used to repel charged tether ends of the single or double stranded DNA (ligands or ligand/antiligand complexes) from the first substrate to stretch them towards the second substrate.

Multiple generations of replica arrays may be produced by hybridization transfer printing, alternating between target and probe (ligand and antiligand) arrays. If there is a low practical limit to the number of complementary replicas that can be printed from a single master array, then a large number of replica arrays can be made nonetheless by producing as many complementary replicas as possible in a first generation, then as many complementary replicas as possible from each of those replicas in a second generation, and so on.

In the practice of the present invention, a master array of probe DNA can be formed onto the tympanic sensor array described below. Alternatively, the master ligand DNA array can be replicated by hybridization transfer printing to form a second-generation anti-ligand array, and the second-generation complementary anti-ligand array can then be used as a master and replicated to form a right-reading, third-generation anti-complementary replica of the master array essentially identical to the original master array of ligand DNA. It is convenient herein sometimes to refer to the complementary replica made by hybridization transfer as a “reverse replica” of the master from which it is made.

C. Biomolecules Other than DNA

Any substances that, like DNA, hybridize or bind preferentially and reversibly to complementary substances or to the same substances, may be used in Hybridization Array Replication. For example, antibodies can be used to make a master array while corresponding antigens are used to make complementary arrays; or antigens can be used to make a master array while antibodies can be used to make a complementary array. In fact, because antigens such as microbes or spores are much more massive than the antibodies they would bind to, and because the tympanic sensor is a mass change sensor, the sensor of this invention should be much more sensitive for the detection of microbes and spores than for the detection of specific DNA sequences.

Antibodies specific to other antibodies may be used in this invention. For example, an original array may be made using a set of 100 pathogenic microbes, and a complementary replica array may be made from the original by using a first mixture of 100 different antibodies specific to each of the 100 microbes. A further generation of replicas may then be made by using a second mixture of antibodies specific to each of the microbe-specific antibodies. Although the antibodies in the second mixture are different from those in the first mixture, and may hybridize with less or more specificity than the antibodies in the first mixture hybridize to the microbes, antibodies in the first and second generation replica arrays are complementary in the sense necessary to carry out this invention. That is, the second-generation antibody array is in a sense equivalent to the microbe array, so elements in the third-generation antibody arrays made from second-generation arrays are capable of binding with a useful degree of specificity to the corresponding microbes in the original array.

D. Attaching Biomolecules to a Substrate

A technique widely used in the biotechnology industry for binding DNA to gold begins with commercially available DNA oligomers modified to carry a thiol group on one end of the molecule. The thiol group attaches easily and selectively to gold. A published M.Sc. thesis by Rodolphe Marie, which is incorporated herein by reference, from Mikroelektronik Centret, MIC Technical University of Denmark, DTU September 2000; corrected version January 2002, entitled “DNA HYBRIDIZATION INVESTIGATED BY MICROCANTILEVER-BASED SENSOR” specifies the following steps to “deprotect” the thiol group in order to provide more efficient binding to gold:

Appendix A. Protocol of deprotection of thiol modified DNA oligos

1. Suspend the oligos in a 0.1M triethylammonium acetate (TEM) solution pH 6.5 at a concentration of approximately 100 A260 units/ml (defined below).

2. Add 0.15 volumes of 1M aqueous silver nitrate solution, mix throroughly, and leave at room temperature for 30 minutes.

3. Add 0.15 volumes of 1M aqueous ditreithiol (DTT) solution, mix thoroughly, and leave at room temperature for 5 minutes. Observe a pale yellow precipitate.

4. Centrifuge the suspension to remove the silver DTT complex.

Remove the supernatant. Wash the precipitate with 1 volume of 0.1 M TEM. Centrifuge and combine the second supernatant with the first one. (One A260 unit=4.2564 μM in the case of the 25′mer ss-DNA oligos used because this unit is related to the mass of the oligos.)

Also published in the same M.Sc. thesis is a very simple protocol for covalently binding thiol-terminated DNA to gold. The most important element of the protocol is to either prevent freshly deposited gold-from coming into contact with carbon dioxide in the air, or to clean the gold surface before exposing the gold to the thiol-terminated DNA. Whereas Rodolfe Marie specifies cleaning gold with aqua regia, another approach ensures cleanliness by coating gold onto a substrate in a vacuum chamber and then filling the vacuum chamber with carbon dioxide-free inert gas such as nitrogen, and keeping the substrate in an inert atmosphere for all subsequent handling until the covalent binding is completed. To accomplish the covalent thiol/gold binding, it is only necessary to bring the clean gold surface into contact with an aqueous solution of the deprotected thiol-terminated DNA for about 5 minutes.

Hybridization of “capture” [probe] DNA to complementary “target” DNA may be accomplished by the following protocol:

1. Rinse substrate with its covalently bound “capture” DNA in Dl water for 5 minutes

2. Run 5×SSC solution over the substrate for a few minutes (5×SSC is a solution of sodium chloride (0.75M), sodium citrate (0.075M) and detergent (0.1% in vol. of Tween20). Tween20 may be purchased from Merk-Schuchardt, and 20×SSC solution may be purchased from Promega. 20×SSC is an aqueous solution of sodium (3M) and sodium citrate (0.3M). 5×SSC is a dilution of this 20×SSC solution.

3. Run 10 uM target DNA solution over the substrate

4. Rinse in Dl water.

5. Dry in warm flowing air.

There are alternative methods described in the scientific literature for immobilizing DNA or proteins on surfaces, and the following publications incorporated herein by reference. J. H. Kim et al (Journal of Biotechnology 96 (2002) 213-221) compare methods using thiolated oligomeric ssDNA, one containing a disulfide bond and another containing a sulfhydryl group. T. Strohter et al (Nucleic Acids Research, 2000, Vol. 28, No. 18 3535-3541) describe using a UV-mediated reaction to attach either the 3′ or the 5′ end of a ssDNA strand to silicon surfaces. A. Kkumar et al (Nucleic Acids Research, 2000, Vol. 28, NO. 14) describe the use of silanized DNA to immobilize DNA onto glass surfaces. M. Beier and J. Hoheisel (Nucleic Acids Research, 1999, Vol. 27, No. 9, 1970-1977) describe a linker-based procedure that allows covalent bonding of DNA to glass or polypropylene surfaces, and allows up to 30 cycles of hybridization and stripping with no apparent loss of signal. Some researchers such as R. McKendry et al (PNAS Jul. 23, 2002, Vol. 99 No. 15, 9783-9788) have used a titanium adhesion layer with a gold overlayer to adhere thiolated DNA to silicon surfaces. M. Kane et al (Nucleic Acids Research, 2000, Vol. 28, NO. 22, 4552-4557) described using a 5′ amino linker on DNA to allow covalent attachment to Surmodics 3D-Link activated slides. K. Lindroos te al (Nucleic Acids Research, 2001, Vol. 29, No. 13 e69) attached disulfide-modified DNA to mercaptosilane-coated glass slides. G. Lee et al (Science 266, Nov. 4, 1994, 771-773) covalently attached thiolated DNA to self-assembled monolayers of gamma-aminoprophylaminoethyltrimethoxysilane on silica surfaces by using succinimidyl 4-[P-maleimidophenyl]butyrate. R. Penchovsky et al (Nucleic Acids Research, 2000, Vol. 28, No. 22 e98) described a method for attaching 5′ amino-modified DNA to both silica and polystyrene surfaces using a photo-reactive cross-linking chemical, 4-nitrophenyl 3-diazopyruvate.

II. Tympanic Sensor Array

The sensor array of the present invention is an adaptation of a Membrane Light Modulator (MLM) described by Preston (Proc. ISSCC, 100 (1968), Kendall Preston Jr., An Array Optical Spatial Phase Modulator) (FIG. 2) and by Reizman (Proc. Electro-opt. Syst. Design Conf., New York September, 1969) (FIG. 3), both references being incorporated herein by reference. The purpose of Preston's and Reizman's MLMs was to spatially phase-modulate light; and neither Preston nor Reizman made any suggestion that their MLMs could be used for any purpose other than light modulation. In the present invention, the MLM is not used for light modulation.

A. Construction

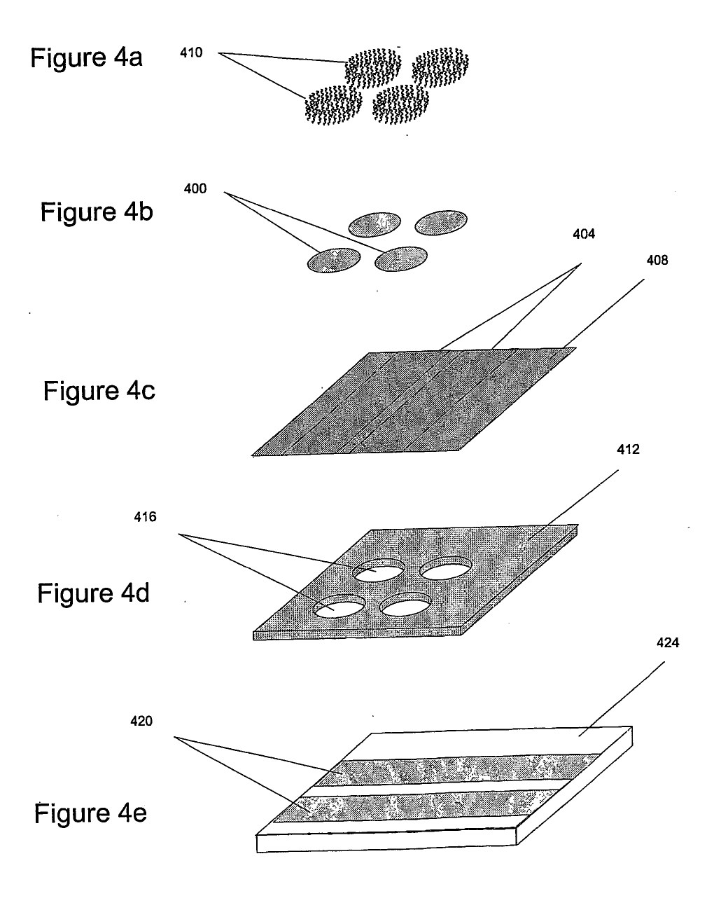

In the present invention, a structure very similar to that of an MLM is fabricated as indicated in FIGS. 4 through 4e.

In a first step illustrated in FIG. 4e, electrode stripes 420 are applied to a substrate 424 such as glass, silicon or polyester, for example by the well known method of vacuum evaporation of gold onto the substrate, followed by photoresist coating, photo-exposure through a mask, photoresist development and etching away the gold from the portions then no longer covered by photoresist.

In a second step illustrated in FIG. 4d, a thin, hole-containing (perforated) layer 412 is applied to the substrate 424 by a method such as UV casting described in U.S. U.S. Pat. Nos. 4,758,296 and 4,906,315, incorporated herein by reference. The holes 416 in the hole-containing layer are aligned precisely with the electrode stripes 420.

In a third step illustrated in FIG. 4c, an elastic polymer membrane 408 such as collodion is adhered to the hole-containing layer so that it covers each hole 416.

In a fourth step illustrated also in FIG. 4c (which may be performed before or after the membrane is adhered to the hole-containing layer), electrode stripes 404 are applied to the top surface of the membrane 408. The electrode stripes on the membrane surface are oriented at right angles to the electrode stripes 420 on the substrate 424.

In a fifth step illustrated in FIGS. 4a and 4b, a complementary DNA array 410 is applied to the electrode stripes 404, such that the distinct regions 400 containing distinct kinds of DNA 410 lie on the membrane over the holes 416 in the hole-containing layer. The fifth step may be done prior to or after adhering the membrane 408 to the hole-containing layer.

The substrate 424, a hole 416, and the membrane 408 over the hole constitute a “drum”, with the membrane serving as the “drumhead” or “tympanum” (hence the name, “tympanic sensor”). An electrical voltage applied to the two electrode stripes 404 and 420 that constitute the top and bottom of the “drum” causes the drumhead to be deflected toward or away from the bottom of the hole due to electrostatic attraction or repulsion. An oscillating voltage will cause the drumhead to vibrate at the frequency of the oscillating voltage, with a phase and amplitude dependent on the difference between the driving frequency and the resonant frequency of the drumhead. The resonant frequency of the 5 drumhead depends on the mass of the drumhead as well as the elasticity of the membrane and the compressibility of any fluid or gas that may be inside the drum. When complementary target DNA binds to the probe DNA strands attached to the electrode on the membrane forming the drumhead, the resonant frequency will decrease due to change in the mass of the drumhead. The resonant frequency can be measured electrically by driving the drumhead with a “chirped” signal, an oscillating voltage whose frequency of oscillation changes over time. The electrical impedance of the drum will reach an extreme at the resonant frequency.

Thus, the present invention includes a method for “printing” complex DNA arrays onto surfaces by Hybridization Transfer Printing, and a sensor array structure and method for electrically detecting mass changes on a membrane drumhead due to complementary DNA hybridization.

Instead of DNA, any substance or set of substances may be used that can bind specifically and reversibly to a corresponding complementary substances or set of corresponding complementary substances. For example, antibodies and antigens, or antibodies and specific anti-antibodies may be used. Methods for producing antibodies specific to particular antigens are well known in the art, as are ways to produce DNA complementary to other DNA. In some cases the target and probe substances may be identical. For example, many substances such as sugars and salts preferentially and reversibly bind to molecules similar or identical to themselves, so that a solution containing a mixture of such substances will self-organize into a collection of pure crystals, each crystal containing only one such substance.

B. Sensitivity

A 38-micron diameter “drum” sensor with a 0.1 micron thick collodion membrane “drumhead” typically has a resonant frequency in the range of 1 to 10 MHz. Thus, such a sensor will be able to detect mass changes on the order of a millionth of the drumhead's mass, given a reading time of one second.

The mass of such a drumhead is on the order of 100 nanograms, with a mass density roughly comparable to that of densely packed DNA, and a thickness of about half a micron or 500 nanometers. A single-stranded DNA molecule 20 nucleotides long is approximately 70 nm long, so the mass of a dense coating of complementary DNA on the drumhead would be about 14 nanograms. Thus, the effective mass of the drumhead would increase by an easily detected factor of a few percent due to hybridization with complementary DNA.

Microbes or spores in the size range of 1 to 10 microns binding to the drumhead would change its mass by a factor of 2 to 20, so will produce a very large change in the resonant frequency of the drumhead.

C. Manufacture

In a preferred embodiment of the invention, a DNA sensor array is manufactured in two stages: first a deformable membrane sensor array is manufactured, and then DNA is selectively applied to the sensors in the array.

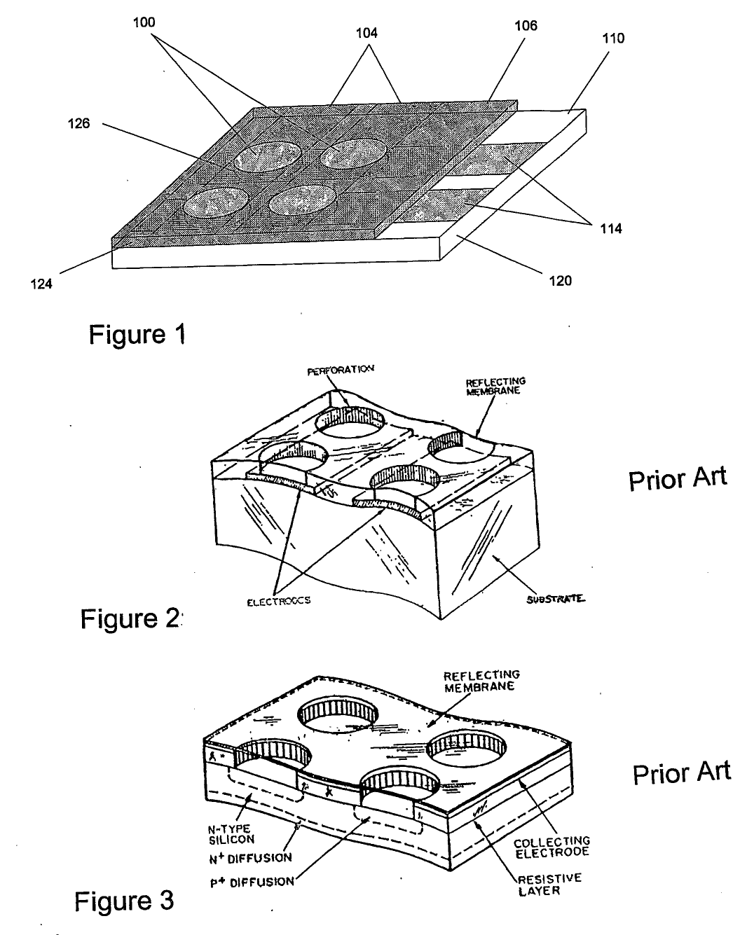

The preferred embodiment of the sensor array in the present invention is illustrated in FIG. 1. A low-cost, high-speed manufacturing process for the tympanic sensor arrays includes A) a web-based system to manufacture a perforated layer on a substrate film bearing a first pattern of stripe electrodes in one direction so that the stripe electrodes are directly in register with rows of perforations, B) a system to apply a thin elastic membrane to the perforated layer, and C) a system to apply stripe electrodes in a second pattern to the membrane in register with columns of perforations in the perforated layer so that the electrodes in the second pattern and the first pattern cross at the perforations in the perforated layer.

It is preferred but not necessary that the second pattern of stripe electrodes are on the side of the membrane opposite to the perforations in the perforated layer, so that DNA may be attached selectively to the membrane electrodes at the location of the perforations as indicated in FIG. 1 and FIG. 2. However, it is also possible to apply electrodes to the membrane before applying the membrane to the perforated layer, so that the electrodes are between the membrane and the perforated layer. In that case, DNA can subsequently be attached selectively to regions of the membrane directly aligned with the perforations if the regions are primed to allow attachment of the DNA.

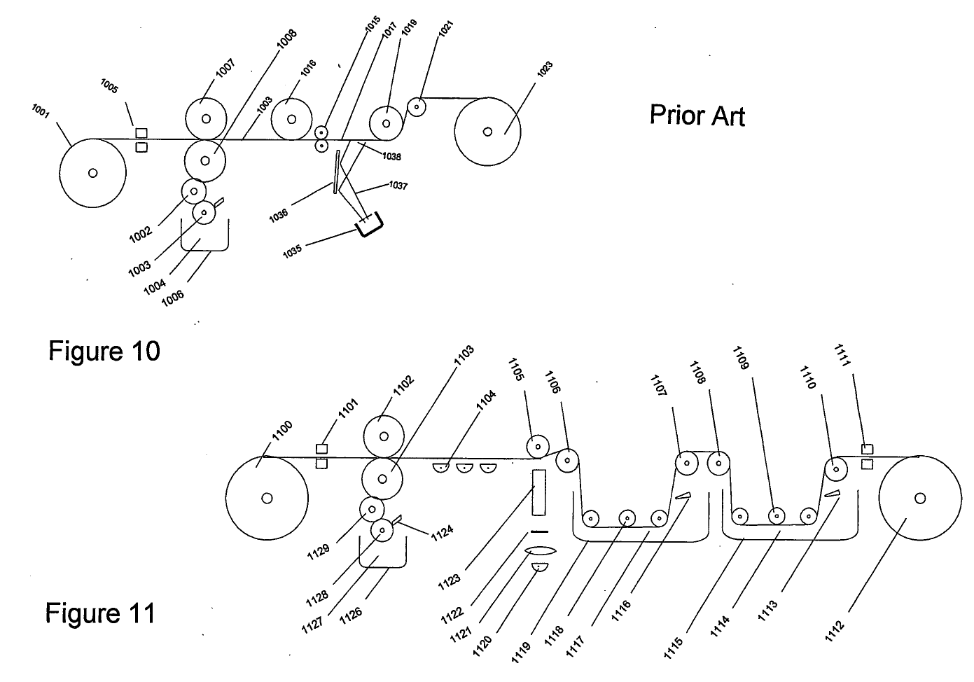

The sensor array is preferably manufactured in several steps as illustrated in FIG. 10a through FIG. 10c.

1. As illustrated in FIG. 10a, print Fomblin oil from reservoir 1006 onto polyester film web substrate 1003 in lateral (across the web) stripes using gravure printing cylinder 1008. Fomblin oil is widely used in oil diffusion pumps and is readily commercially available. The Fomblin oil prevents adhesion of sputtered or evaporated substances in the regions to which the oil is applied. Alternative embodiments may use stripes or other patterns oriented in directions other than right angles to the web direction and may have different shapes and sizes. An edge guide system can use edge sensor 1005 to ensure correct registration to the edge of the web or to features that pre-exist on the web.

2. Using an ion gun 1035 and ITO target 1036. Sputter indium tin oxide (ITO) in a vacuum onto the surface of the film (at 1038), to a thickness of 800 Angstroms. No ITO adheres in the oil-printed regions. Vapor deposition and other methods may be used instead of sputtering, and any transparent or opaque electrically conductive material may be used instead of ITO.

3. Drive the oil off by heating, so that ITO remains on the film where there was no oil printed, and the oil-printed parts of the film have no ITO. For example, this may be accomplished by running the polyester web under a heated roller 1019.

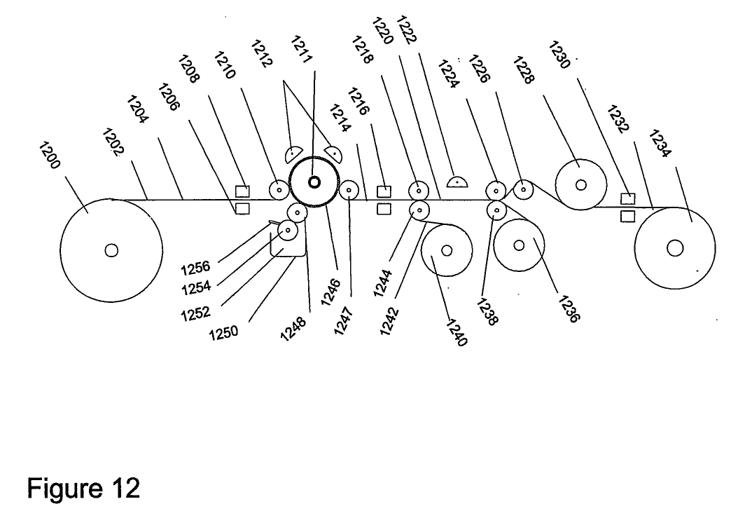

4. Using a gravure coating cylinder 1248 in FIG. 12, coat a UV-curable resin such as Series 108 UV-Curing Conformal Coatings from Crosslink Technologies, Inc. onto a drum 1246 bearing raised portions corresponding to the desired locations of perforations in a perforated layer. The raised portions for example may be cylindrical, 2 microns high and 38 microns in diameter; and the surface of drum 1246 may be composed of nickel. It is advantageous to apply UV resin in a layer a few microns thick, only in the regions where the perforated layer is desired. In particular, it is preferable to leave a region uncoated with UV resin, so that it is easy to make electrical contact with the ends of stripe electrodes 114 and 104 as indicated in FIG. 1.

5. Press the polyester film against the UV resin on drum 1061 using nip roller 1210 and cure the resin through the substrate using UV light from a mercury discharge lamps 1212 as indicated in FIG. 12. If instead of ITO, an opaque electrically conductive material such as gold is used, then the resin may be cured through a UV-transparent drum 1246 instead of through the substrate, using UV lamp 1211. Register the pattern on the drum to the stripe electrode pattern on the film so that the raised portions on the drum are directly under the stripe electrodes. Preferably, the resin is only partially cured in this step. The partially cured resin is at this point in the form of a perforated uniform layer approximately 2 micron thick, with 50-micron diameter round perforations spaced 75 microns center to center over the stripe electrodes. The resin should be cured enough that it resembles an elastic solid, but still has some unpolymerized, mobile oligomer in it. Following the partial cure, remove the web from the drum 1246 using nip roller 1247. The resulting structure is a polyester substrate with lateral stripe electrodes, covered by a perforated layer whose perforations are aligned with the stripe electrodes.

6. Laminate a 0.1 micron collodion membrane (carried on a polyester film web 1242) as shown in FIG. 12 onto the perforated layer using lamination roller 1244. Complete the curing of the UV resin by further exposure to UV light using UV lamps 1222 in order to firmly bind the membrane to the perforated layer while leaving the tympanae unsupported except at their edges, and remove the polyester carrier film using nip roller 1238.

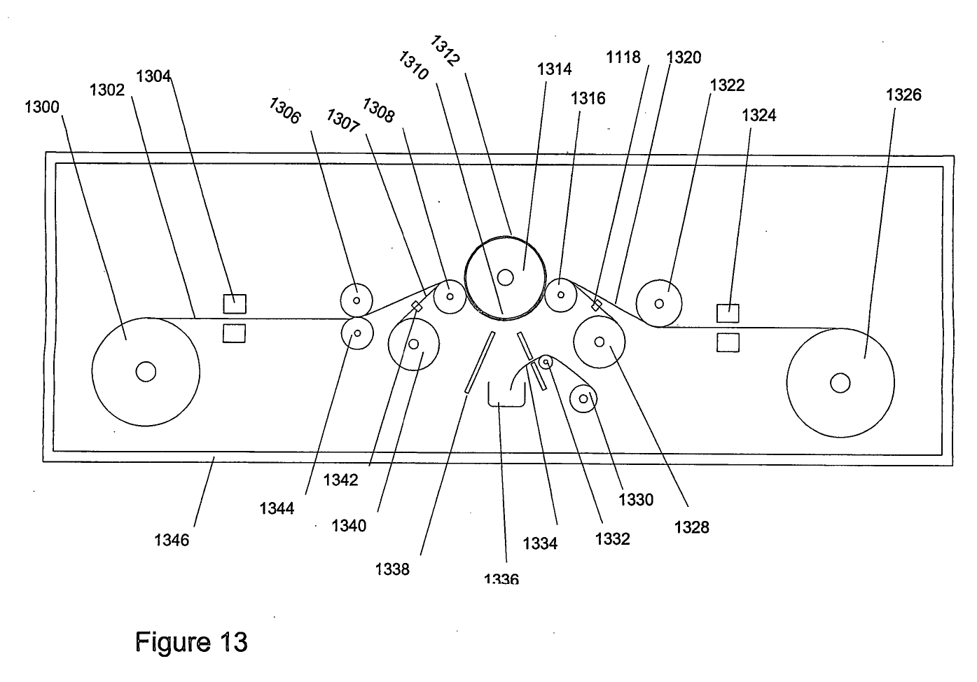

7. As shown in FIG. 13, evaporate 500 Angstroms of gold in longitudinal stripes onto the collodion membrane in register with the longitudinal rows of perforations in the perforated layer to which the collodion membrane is laminated. The membrane may be masked as shown in FIG. 13 by an array of 50-micron diameter wires 1307 fed through guide holes 1342 spaced 75 microns apart along a line. A feed spool 1340 and a take-up spool 1328 move the wires at a constant speed so that gold evaporated from boat 1336 fed by gold wire 1334 does not build up thickly enough on the wires to substantially change their diameters during the gold deposition.

At the conclusion of this Step 7, the web consists of an array of drum-like structures (perforations) with stripe electrodes across the web at the tops of the structures on the polyester substrate and stripe electrodes along the web at the bottoms of the structures on the thin and flexible collodion membrane. A voltage between the top and bottom electrodes of any one of the drum structures will cause an electrostatic force between the electrodes, which results in deflection of the membrane. A given pair of electrodes on the top and bottom of the array will deflect the membrane primarily in only the one such structure where the pair of electrodes cross.

Applying Probe DNA to the Tympanic Array

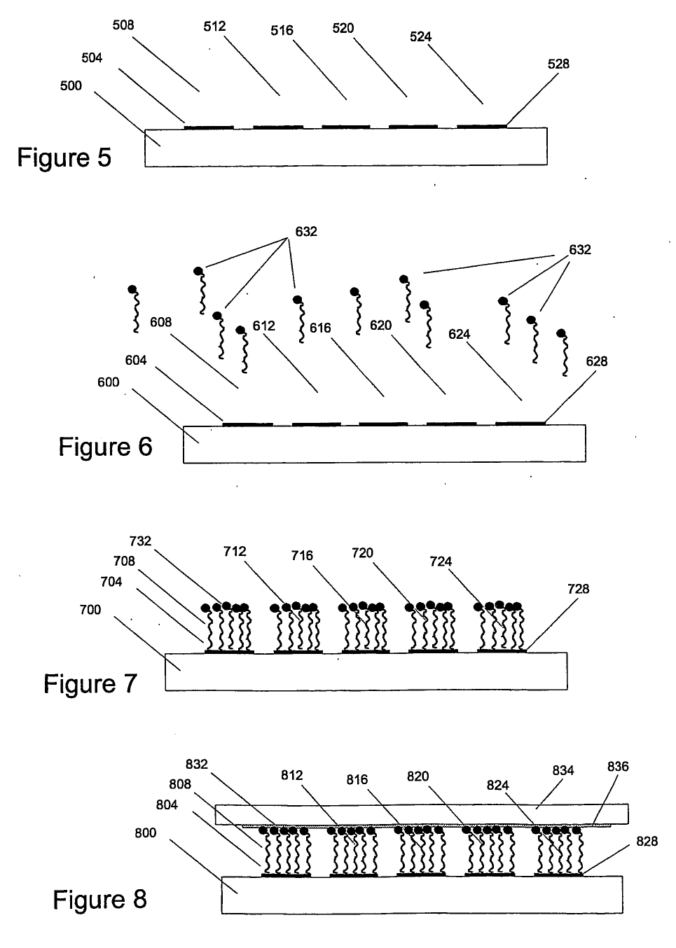

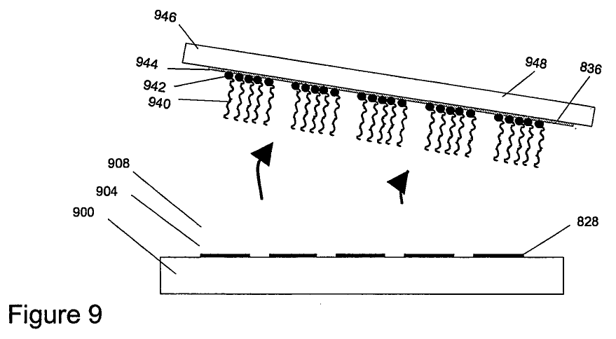

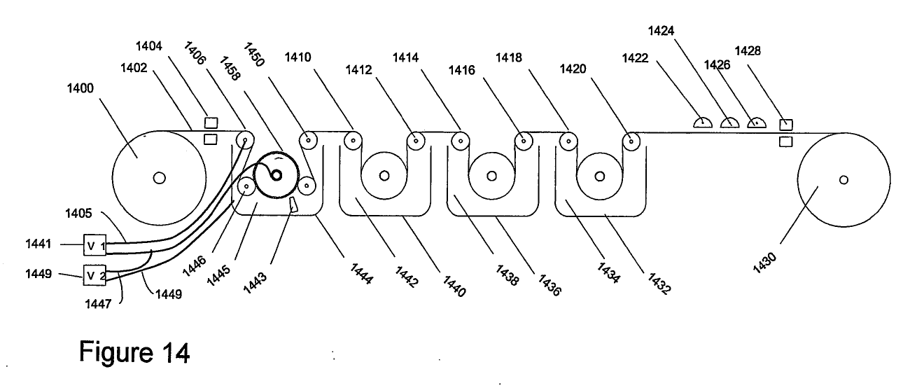

8. Prepare a master array of target DNA oligomers on a cylinder 1458 in FIG. 14, using for example the process illustrated in FIGS. 5, 6, 7, 8, and 9 to produce a master array on a flexible film substrate which is then adhered to the outside of the cylinder 1458.

9. Hybridize a mixture of probe DNA strands with the DNA on the master array by exposing the cylinder 1458 to a solution 1445 containing the probe DNA mixture. The probe DNA strands are preferably terminated by thiol groups, but may alternatively be terminated by nanocrystals, magnetic beads, or any other ends that facilitate the binding in Step 6.

10. Bind the probe DNA ends to a second substrate 1402 in close proximity to the first substrate on master cylinder 1458. A preferred way to do this, shown in FIG. 14, is to press a flexible substrate 1402 against the master array 1458 (with the flexible substrate being first gold coated in spots registered to the elements of the master array) in the presence of suitable chemistry so that the gold on the flexible substrate binds covalently to the thiol ends or terminations on the probe DNA. For example, unprotected thiol groups in aqueous solution bind spontaneously to gold on a substrate at room temperature as described by Rodolfe Marie. The flexible substrate 1402 may be, for example, a collodion membrane carried on a polyester film and pre-coated with gold stripe electrodes.

11. Separate the two substrates to leave a probe DNA array bound to the film substrate, complementary to the master array. This step is preferentially done by heating the two substrates to cause “melting” or unbinding of the double-stranded DNA formed by the hybridization of DNA on the master array with target DNA. The heating may be done intermittently as the film is advanced in steps, or it may be done continuously as the film slowly advances by pumping the solution 1446 in tank 1444 through an appropriate arrangement of heaters, chillers, and/or heat exchangers and expelling the heated mixture through jets 1443 onto the back side of the film. Alternatively, heating and cooling elements in the interior of cylinder 1458 may be used to maintain stationary warm and cool zones on the cylinder surface while the cylinder turns. The probe DNA stays attached to the flexible second substrate 1402, leaving the master target array DNA still attached to the original substrate 1458. However, the physical arrangement of the DNA on each substrate remains substantially fixed due to the strong chemical bonds between DNA and substrate, via the thiol ends.

In FIG. 14, three separate modules are shown for further sub-steps such as rinsing; and a drying subsystem 1422, 1424, 1426 and edge guides 1428, 1404 are also shown.

The patterning methods described here, using printed Fomblin oil and using a fiber mask, are only two of many alternative patterning methods that can be used to pattern electrodes on the substrate and the membrane, such as photolithography, pad printing, evaporation through a mask, and laser ablation. Similarly, any suitable method may be used for forming the perforated uniform layer, such as photolithography, laser ablation, thermal molding, or laminating a mechanically perforated film onto the film substrate. The film substrate can be polyester or any other flexible or rigid material, though a material with dimensional stability is preferable. The membrane material is preferably a self-assembled monolayer film such as a Langmuir-Blodgett layer, because such films typically have extremely uniform and repeatable thickness and mechanical properties.

Registration accuracy, at least great enough to align the electrodes, perforations and DNA array spots, is important in making high-quality, high-density DNA array sensors according to this invention. If electrodes are pre-patterned on the membrane, one way to manage the task of registration is to apply tension to slightly stretch the membrane as it is laminated to the perforated layer, and vary the amount of tension via feedback from a registration sensor. The registration sensor may be an optical registration sensor to detect the position of the electrodes or to detect the position of other markings applied to the film, perforated layer or membrane.

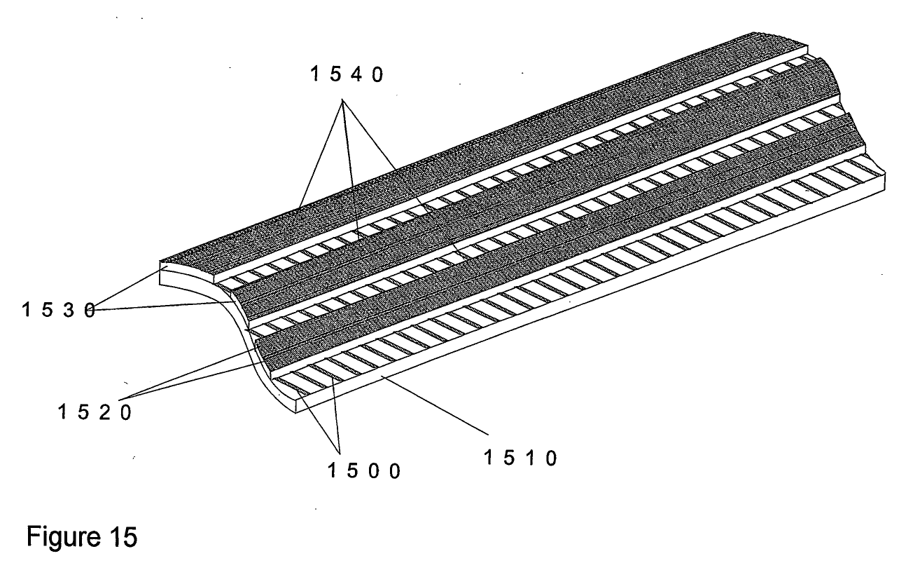

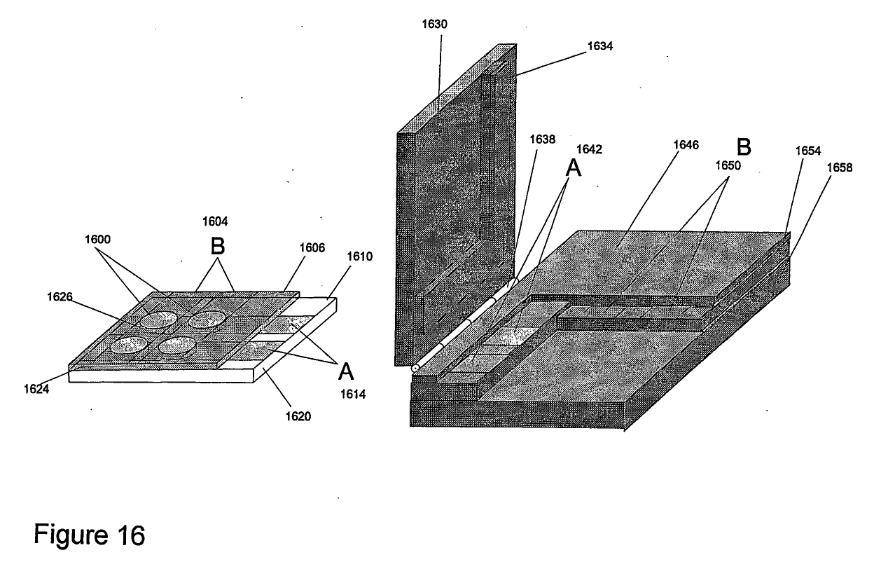

In the steps described above for manufacturing tympanic DNA hybridization sensor arrays, a continuous roll of sensor arrays is produced, like that shown in FIG. 15, for example, as a polyester or other semi-rigid substrate film 1510 bearing lateral stripe electrodes 1500 overlain by perforated layer 1530 which is in turn covered by thin elastic membrane 1520 bearing longitudinal electrodes 1540. Individual hybridization sensor arrays are cut from the roll by any suitable means such as laser cutting or die cutting. A finished hybridization sensor array may contain as few as one sensor element or as many as hundreds of thousands of sensor elements. In any case, means are provided to read changes in the mass of each drumhead. In the embodiment illustrated in FIG. 1, the drumhead mass changes due to hybridization with target DNA are read out by making electrical contact with the lateral and longitudinal stripe electrodes 114 and 104, so the array is fabricated in such a way that both lateral and longitudinal stripe electrodes are uncovered each along at least one edge of the “chip”. A preferred read out system is illustrated in FIG. 16. Electronics module 1645 makes electrical contact with the stripe electrodes on the chip such that chip electrodes 1614 marked “A” make contact with electrodes 1642 also marked “A” on the readout device, and chip electrodes 1604 marked “B”; make contact with electrodes 1650 also marked “B”.

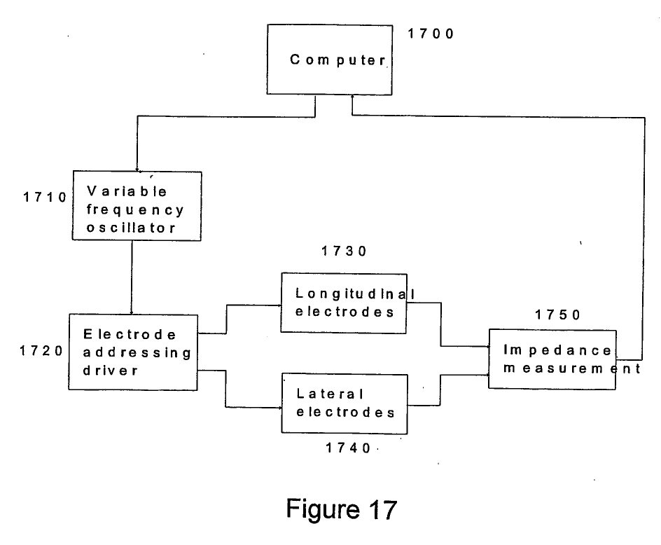

The electronics module 1654 performs the functions outlined in FIG. 17. Under control of a computer 1700, an oscillator provides a series of different frequencies to a sensor element in the array through the electrodes 1730 and 1740 by means of the electrode addressing driver 1720, and measures the impedance of the sensor at each frequency by means of the impedance measurement module 1750. The computer 1700 then compares the impedance measurements to an internally stored model in order to discern any change in the resonant frequency of the sensor.

It is preferable to use gold for the stripes on the membrane layer, and collodion for the membrane layer, but the stripes on the polyester film substrate can be gold, silver, copper, indium tin oxide, or any other electrically conductive substance that adheres well to the film. Similarly, the membrane layer can be polyester, PVC, collodion, OPP, or any other substance that forms an elastic thin film, can be adhered to the perforated layer, accepts an electrically conductive layer and is not damaged by substances used in attaching DNA to the membrane layer or hybridizing complementary DNA to the membrane-bound DNA.

In some embodiments of the invention, the membrane may be composed of two layers having different thermomechanical, photomechanical or electromechanical properties, so that heating, illuminating or applying a voltage across the layers causes a mechanical deflection of the membrane similarly to the way a bimetallic strip in a thermostat deforms when its temperature changes. For example, the membrane may be composed of two piezoelectric or electrostrictive materials. In that case, the appropriate excitation energy may be applied directly to the membrane at a desired drumhead to cause deflection, at several oscillatory frequencies different from the resonant frequency of the drumhead. The resulting oscillation amplitudes may be used to calculate the resonant frequency according to the well-known physical principles governing a driven, damped harmonic oscillator.

The perforated layer may be pre-formed and laminated to the substrate instead of cast in place on the substrate. Alternatively, the perforated layer may be thermo-formed by casting, extruding or otherwise applying a thermoformable plastic layer onto the substrate and pressing a heated die against the thermoformable side with a pressure roller on the other side.

The method for laminating the membrane to the perforated layer may include taking advantage of the tackiness of a partially cured UV resin as described above, or it may use an adhesive layer thermal bonding, or any other means to ensure adhesion between the membrane and the perforated layer.

In the steps outlined above, antibodies or antigens may be used instead of DNA and complementary DNA, thereby making a master antigen array and subsequently making antibody arrays from the master antigen array, or by making a master antibody array and subsequently making antigen arrays from the master antibody array. Similarly, RNA sequences complementary to DNA sequences may be used, and vice versa. Analogues to biomolecules may be used rather than actual biomolecules, to control binding strengths, hybridization/dehybridization temperatures (Tm) binding specificity, and so on without departing from the scope of this invention. Throughout this disclosure and in the claims, when DNA and complementary DNA are mentioned, it is to be understood that any pairs of substances can be used that selectively bind reversibly to each other, can be strongly bound to a substrate, and can be separated from each other without unbinding them from their respective substrates.

Whereas gold may be used as both an electrical conductor and a surface that DNA can be bound to, it is possible to use other electrical conductors such as ITO to form the stripes on the elastic membrane, either in conjunction or not with an additional surface coating to which DNA can adhere.

The substance arrays and sensors do not necessarily have to be made on a roll-to-roll basis, and may be made on flat sheets as indicated in FIG. 5 through FIG. 9.

A voltage may be applied between the DNA solution and a substrate to electrophoretically increase the density of DNA at the substrate and thereby speed the rate at which DNA binds to the substrate. Similarly, when a first substrate bearing tethered single-stranded DNA with complementary DNA hybridized to it is brought into close proximity to a second substrate, a voltage between the first and second substrates can effectively lower the melting temperature for de-hybridization and speed the attachment of the complementary DNA strands to the second substrate. Thus, in the roll-to-roll process for replicating DNA arrays, it is advantageous to provide means 1449 for applying a voltage between the master drum and the surrounding fluid, and means 1441 for applying a voltage between the master drum and the electrodes on the membrane drumheads.

These and other prior-art methods may be used within the context of the present invention to bind DNA firmly to a master array substrate, as well as to bind and then transfer complementary DNA to a second substrate after hybridization to the master array.

Operation of the Sensor

The finished array sensor is illustrated in FIG. 1. In FIG. 17 the electrical readout system is schematically illustrated. Under the control of a computer 1700, an oscillator 1710 provides a signal via electrode addressing driver 1720 to individual sensor elements 1600 (FIG. 16) corresponding to row/column addresses. The impedance before and after hybridization, at several different oscillation frequencies, is measured by impedance measurement module 1750. Computer 1700 determines the mass change in each drumhead in the sensor array by comparing impedances before and after hybridization, using a mathematical model of the dependence of frequency dependent impedance on drumhead mass. A typical tympanic structure such as a sensor element in FIG. 1 is electrically equivalent to an LC circuit and has a resonant frequency on the order of 1 to 10 MHz, though the frequency may be higher or lower depending on the temperature of the membrane, interactions between the membrane and any fluid that it is in contact with, and the membrane's thermal or chemical history. The resonant frequency depends on the mass, size and elastic coefficient of the drumhead (the collodion membrane, plus the gold and DNA bound to it).

The mass densities of collodion and tightly packed DNA are roughly the same. The collodion membrane may have a thickness of 100 nanometers, and the gold layer may have a thickness of about 50 nanometers, with a mass density approximately four times greater than collodion. The original DNA layer has a thickness of about 70 nanometers, but its effective mass density is doubled when complementary DNA is hybridized with it. Thus, regardless of the lateral size of the structure, when complementary DNA hybridizes with the original DNA the change in mass of the drumhead is on the order of a few percent. The resonant frequency of the structure is approximately inversely proportional to the mass of the drumhead, so the change of resonant frequency of the structure typically will be in the range of 1 to 10 percent as well. Thus, the change in mass can be estimated by the change in resonant frequency of the structure.

If the elasticity of the collodion membrane changes, the resonant frequency and impedance will change as well. This can occur as a result of solvent or chemical interactions between a sample and the collodion membrane, or as a result of temperature or humidity change. Thus, a preferred sensor will include reference drumheads bearing either no original DNA at all or a DNA sequence that either is very unlikely or is certain to be complementary to reference DNA found in the sample to be tested. The resonant frequency or impedance change of the reference drumheads may then be measured before and after the sample DNA is added for hybridization. Any change in the elasticity of the collodion membrane drumheads will be nearly the same for all drumheads, so the important parameter to measure is the change of impedance or resonant frequency of each sensor drumhead relative to the reference drumheads that have been exposed to the same thermal and solvent history as the DNA-coated drumheads.

A preferred way to sense changes in resonant frequency of a given drumhead is to apply a variable-frequency electrical signal between the row stripe and the column stripe that cross at that drumhead, in a frequency range that is near the resonant frequency of the drumhead. The impedance is measured at three or more frequencies, and the frequency measurements are fitted to an impedance curve family parametrized according to drumhead mass and elasticity.

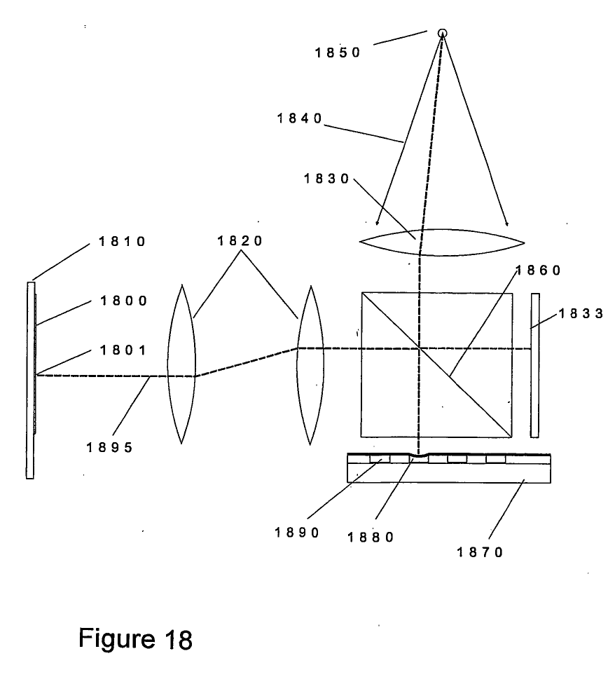

FIG. 18 illustrates an optical read-out system for a tympanic sensor array 1870. A coherent light beam 1840 emitted from source 1850 is collimated by lens system 1830. Beam 1840 is divided into two components by beamsplitter 1860. One component reflects off the beamsplitter, reflects off mirror 1833, and is imaged onto photodetector array 1800 by lens system 1820 after passing through beamsplitter 1860. The other component passes through beamsplitter 1860, reflects off the tympanic sensor membrane, reflects off beamsplitter 1860, and is imaged onto photodetector array 1800. The two beams interfere on the photodetector array 1800, one carrying a phase image of the membrane surface of tympanic sensor array 1870 (essentially a distorted flat wavefront) and the other carrying an image of the mirror 1833 (essentially a featureless, flat wavefront). If the position of mirror 1833 is adjusted to produce a pattern of dark spots corresponding to the tympanic elements of the array 1870 when there is no voltage applied to the tympanae, then application of a voltage deflects the tympanae, producing an array of bright spots.

When an oscillatory voltage near the resonant frequency of a tympanum is applied, the tympanum oscillates at the driving frequency but with a phase difference depending on the difference between the driving frequency and the resonant frequency of the tympanum. The resonant frequency corresponds to the mass of the tympanum; the mass of the tympanum corresponds to the hybridization state of the probe species on the tympanum's surface; and therefore the hybridization state may be inferred from the phase difference between the driving frequency and the oscillation of the brightness of the image spot corresponding to the tympanum of a sensor element 1880 or 1890.

Changes in the mass of a drumhead, with consequent changes in the resonant frequency of the drumhead, can be caused by dissolution or digestion of substances bound to the drumheads, chemical reactions between substances on the drumhead and substances in a fluid medium, microbial growth, or temperature change, all of which can be detected by the sensor array and readout method described here.

Preferably, the sensor array is operated by applying a solution containing target species, allowing hybridization to occur, and then drying the sensor array before readout. However, in some embodiments the sensor array may be operated in a vacuum or in a liquid medium. In that case, the mechanical behavior of the sensors will be different and the interpretation of the results must be appropriately, modified.

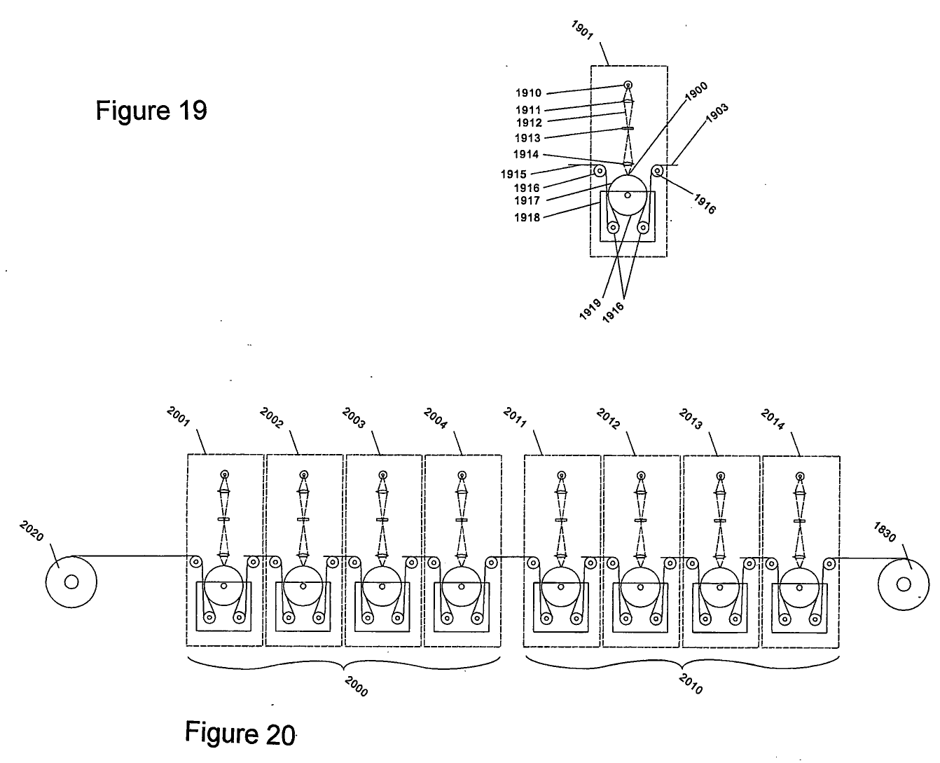

An alternative way to mass-produce DNA arrays according to this invention is by a roll-to-roll photochemical technique. FIG. 19 illustrates one of a series of essentially identical photo-controlled reaction modules in a roll-to-roll photochemical DNA array manufacturing system. A substrate web 1915 passes over rollers 1916 which guide it through the module. The substrate and any DNA bound to the substrate is brought into contact with chemistry in the tank 1918, then over a reactor roller 1919 where the chemistry may be photo-activated by exposure to light 1912 passing through an exposure system consisting of a light source 1910, light concentrator optics 1911, a computer-controlled mask 1913 such as a liquid crystal TV screen or a micromirror array, and imaging optics 1914. Light is focused in the form of a line of light and dark pixels onto the substrate 1917 at a focal line 1900, to activate the chemistry which causes nucleic acids in the tank 1917 to ligate to the ends of DNA strands already bound to the substrate. The photo-controlled nucleic acid binding chemistry is well-known in the art and is disclosed in U.S. Pat. No. 6,379,895 by Fodor, et al., entitled “Photolithographic and other means for manufacturing arrays”, and further disclosed in the references therein. U.S. Pat. No. 6,379,895 and its references are incorporated in the current specification hereby by reference.

III. Photolithoaraphic and Other Means for Manufacturing Arrays

Subsequent rinsing steps (not shown) before and after each photochemical reactor module prepare the substrate 1903 for further photo-controlled nucleic acid ligation steps.

In each module like 1901, a different nucleic acid ligand may be added to DNA strands at loci 1900 on the web according to the exposure pattern at 1900 and the type of nucleic acid in tank 1918. A group of four such modules may add nucleic acid ligands A, G, C, or T to DNA strands at different positions on the substrate, so that every position has one and only one ligand added. A group of 12 such groups of 4 such modules can build any desired pattern of DNA 12-mers on the substrate.

Using a system like that in FIG. 20 to construct DNA arrays “on the fly” using a series of groups 2000, 2010 of modules 2001, 2002, 2003, 2004 and 2011, 2012, 2013, 2014, it is practical to change or customize a DNA array pattern continuously or “on the fly”. A photo-controlled roll-to-roll DNA array manufacturing system such as that in FIG. 20 also has the potential to produce denser arrays with less likelihood of the wrong DNA strands binding to various spots on the array and decreasing the fidelity and reliability of the sensor. As described above, each photochemical reactor module includes any necessary equipment (not shown) for a rinsing step to prepare the substrate for further photo-controlled nucleic acid ligation steps at the next photochemical reactor module.

Among the many applications of this invention is identifying individual human beings or animals according to their individual genotypes. For this, a standard DNA array may be designed, preferentially using the complements to a few hundred highly variable DNA segments from the human genome, DNA segments from the histocompatibility genes, or other gene segments whose combinations vary substantially between individuals. Each individual has his or her own unique pattern of genes, which would result in a unique pattern of hybridization on the sensor array.

For diagnosis of bacterial infections, a DNA array can be prepared with complements to DNA segments unique to specific classes of bacteria, or combinations of genes unique to specific classes of bacteria.

For diagnosis of metabolic disorders, an RNA array can be prepared with combinations of RNA or DNA complementary to mRNA characteristic of those disorders, to with antibodies complementary to proteins characteristic of those disorders.

For diagnosis of genetic disorders, a DNA array can be prepared with DNA segments complementary to DNA segments in the human genome associated with the genetic disorders.

For genetic profiling of a microbial community, a DNA array can be prepared with a full set of DNA 12-mers, or with any set of DNA segments complementary to DNA segments that are salient to distinguishing between different microbial communities.

In the following claims, “species” refers to substances, molecules, polymers, monomers, oligomers, nucleotides, nucleosides, DNA, RNA, proteins, particles, antibodies, viruses, pollens, or antigens that can occur in various types, and to each type of which there is a complementary substance, molecule, polymer, monomer, oligomer, nucleotide, nucleoside, DNA, RNA, protein, particle, antibody, or antigen. “Array” means any regular arrangement of regions. “Species array” means an array of species. “Complementary” means to have a selective, reversible binding affinity as in the case of DNA and cDNA, such that a second species selectively binds to a first species and the binding can be undone by some means such as changing temperature, or illuminating with light. “Complementary species array” means a second species array that is complementary to a first species array in that each region in the second array contains a species complementary to the species in a corresponding region in the first array. A “master species array” is a species array which serves like a template or mold from which species arrays complementary to the master species array can be made. In some cases, a species and its complementary species may be identical. “Hybridization” means the selective binding of a species to its complementary species, and “de-hybridization” means the unbinding of hybridized species. “Electrical impedance measurement” is any measurement or combination of measurements that directly or indirectly measure the electrical impedance or detect changes in the electrical impedance of a thing.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. The word “or” in reference to a list of two or more items, that word covers all of the following interpretations of the word: any of the items in the list, all of the items in the list and any combination of the items in the list. The word “user” refers to a human being or an automatic data processing system that makes use of information or of a system. “Equipment” refers to any material system capable of performing a function, including electronics, machinery, individual human beings, optical systems, or any combination of them.

The above detailed descriptions of embodiments of the invention are not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while steps are presented in a given order, alternative embodiments may perform routines having steps in a different order. The teachings of the invention provided herein can be applied to other systems, not necessarily the depicted system described herein. These and other changes can be made to the invention in light of the detailed description.

The elements and acts of the various embodiments described above can be combined to provide further embodiments. All of the above U.S. patents and applications and other references are incorporated herein by reference. Aspects of the invention can be modified, if necessary, to employ the systems, functions and concepts of the various references described above to provide yet further embodiments of the invention. These and other changes can be made to the invention in light of the above detailed description.

It is claimed:

1. A method for manufacturing an array of biomolecules that have complementary binding species, comprising:
forming on a first substrate, a master-species array composed of a plurality of different species attached to selected regions on the first substrate,
bringing a mixture of the biomolecules complementary to said species into contact with the master species array, such that biomolecules in the mixture bind with complementary species in the master array,
positioning a second substrate into a confronting relationship with the master array, such that biomolecules bound to the master-species array are in proximate confronting relationship to corresponding array regions in the second substrate, and
transferring the biomolecules from the first substrate to confronting array regions in the second substrate, thereby to form an array of biomolecules on the second substrate.

2. The method of claim 1, wherein said biomolecules include a binding moiety selected from sulfhydryl or thiol, effective to react with and bind to the surface of the second substrate, when contacted with the second substrate during said transferring step.

3. The method of claim 2, wherein said biomolecules include a sulfhydryl binding moiety, and the confronting surface of the second substrate includes a gold film.

4. The method of claim 1, wherein said biomolecules are charged at a selected pH, and said positioning includes placing between confronting surfaces of the first and second substrates, a film of electrolyte solution at the selected pH, and said transferring includes applying across the first and second substrates, an electric field whose polarity and field strength are sufficient to cause the biomolecules bound to the first substrate to migrate to corresponding array regions of the second substrate.

5. The method of claim 1, for use in forming an array of different-sequence oligo-or polynucleotides, wherein said species include different-sequence oligonucleotides, and said biomolecules include oligo- or polynucleotides containing at least a segment that is complementary to one of said species.

6. The method of claim 5, wherein said oligo- or polynucleotide biomolecules include a binding moiety selected from sulfhydryl or thiol, and the confronting surface of the second substrate includes a gold film to which the moiety can bind.

7. The method of claim 5, wherein said second substrate is coated with an oligonucleotide having a given sequence to derivatize the surface of said second substrate, and each of the biomolecules bound to the first substrate has a segment whose sequence is common to the other biomolecules and complementary to the oligonucleotide coating the second substrate.

8. The method of claim 1, which further includes repeating the said bringing, positioning, and transferring steps multiple times, thereby to produce multiple arrays.

9. The method of claim 8, wherein the substrate of said master-species array is in the form of a cylindrical drum and the substrate of said complementary species arrays is in the form of a continuous roll of film

10. The method of claim 1, applied to a master-species array that has itself been formed by the method of claim 1, to produce a subsequent generation of complementary replica arrays.

11. The method of claim 1, in which at least one of the processes is done on a roll-to-roll basis with at least one of the substrates being unrolled from a feed roller and re-rolled onto a take-up roller.

12. A sensor array device comprising:
a plurality of individually addressable deformable membrane elements having resonant frequencies that change when mass is added to or removed from the membrane in each sensor,
a readout system that detects changes in the resonant frequency of at least one membrane element in the array, and
a signal-processing system for determining, from detected changes in resonance frequency, the presence of a change in mass of said system.

13. A sensor array device for use in detecting the binding of analyte molecules to one or more array probes, comprising:
a substrate,
an array of vibratory elements formed on said substrate, each element having a vibratory membrane having with a characteristic resonance frequency, when activated by an oscillating electrical field applied to the membrane,
a molecular species probe attached to the membrane of each element array, wherein binding of analyte molecules to a probe is effective to alter the resonance frequency of the associated membrane,
associated with each element, a pair of electrodes by which an electrical field can be applied across the element membrane,
a voltage source operatively connected to said electrodes for applying an oscillatory electric field to a selected element membrane, thereby to induce vibration in the membrane, and
a detector for detecting the frequency or amplitude of induced vibration in an element membrane, thereby to determine the presence or absence of analyte bound to each of one or more membranes in the device.

14. The device of claim 13, wherein the array includes a plurality of rows and plurality of columns of elements, and the pairs of electrodes associated with the elements are formed by a plurality of row electrodes positioned on one face of a plurality of rows of elements, and a plurality of column electrodes positioned on the opposite face of a plurality of columns of elements.

15. The device of claim 14, wherein the voltage source is operable to excite selected row and selected column electrodes, thus to induce vibration in one or more selected elements.

16. The device of claim 13, wherein the voltage source is operable to apply a variable-frequency alternating electrical field across a selected element membrane, and said detector is operable to detect the frequency at which resonance occurs.

17. The device of claim 13, for use in detecting the presence or absence of each of a plurality of different DNA analytes, wherein said probes are different-sequence nucleic acid probes.

18. The device of claim 13, for use in detecting the presence or absence of each of a plurality of different polypeptide analytes, wherein said probes are different-polypeptide-binding probes.

19. An optically-addressed hybridization array sensor comprising:
an array of vibratory elements having a substantially rigid cavity covered by a flexible membrane, said membrane bearing an electrically conductive layer and a probe species layer, and the bottom of said rigid cavity bearing a photo controlled conductive element and electrical contacts attached to an electrical signal source, such that illumination of said conductive element controls an electrical voltage between the bottom or sides of the cavity and the electrically conductive layer of the membrane, and such that the membrane is deflected by an electrostatic force controlled by the electrical voltage; and
an optical addressing system that directs illumination to selected elements of the array at selected modulation frequencies, and a system to detect the response of the drum-like structures to the selected frequencies and infer any change in an amount of target species hybridized to said probe species.

20. An optical read-out system for a hybridization array sensor of the type having an array of deflectable membrane modulators, the resonant frequency of each membrane modulator controlled by hybridization of a target species to a probe species on the membrane of the membrane modulator, said system comprising
a signal source and driver to drive the entire array of modulators at a series of selected frequencies,
an optical phase detection system to measure phase differences between the signal source and the deflection of the modulators at the selected frequencies, and
an information processing system to relate the phase differences to changes in the hybridization state of the membrane modulator.

21. A system for producing chemical arrays on a roll-to-roll basis comprising:
a plurality of exposure modules each comprising a variable masking system, a light source and imaging optics to image a line of pixels from a variable mask to a line on a surface,
a plurality of photochemical modules to bring reactants of a photo controllable chemistry in contact with a substrate bearing reaction loci, such that at each photochemical module is associated with at least one exposure module such that the variable masking system of the exposure module controls illumination of a line of reaction loci on the substrate and thereby controls the photo-controllable chemistry in a spatial pattern of loci according to the variable masking system, such that the plurality of photochemical modules and their associated exposure modules effect an individually predetermined sequence of photo-controlled reactions at each locus on the substrate.

22. A roll-to-roll DNA array manufacturing system comprising a web handling system to move a substrate in the form of a flexible web through a series of groups of four photochemical reactors, each of said photochemical reactors comprising a source of photo-controlled chemistry and an exposure module such that reactive molecules on the substrate are brought into contact with each of four nucleic acids A, G, C, T, in a predetermined order and the exposure module controls the illumination of the substrate at each of a plurality of loci in a line on the substrate substantially perpendicular to the direction of web motion and on the substrate in a predetermined spatial pattern.

23. The method of claim 1, wherein said second substrate comprises an array of vibratory elements formed on said substrate, each element having a vibratory membrane having with a characteristic resonance frequency, when activated by an oscillating electrical field applied to the membrane, and said positioning step includes placing the second substrate into a confronting relationship with the master array, such that biomolecules bound to the master-species array are in proximate confronting relationship to corresponding vibratory elements in the second substrate.

24. A sensor device comprising
a substrate having a plurality of array regions,
attached to each array region, an anchoring nucleic acid covalently attached to each region, said anchoring nucleic acid having a common base sequence, and
hybridized to the anchoring nucleic acid in each region, a nucleic acid probe having a first common-sequence region that is complementary to at least a portion of the anchoring nucleic acid, and a second probe region having a nucleic acid sequence that is different in the different regions.

25. The device of claim 24, wherein the anchoring and common-sequence nucleic acids are covalently coupled to one another.
